(19) United States
(12) Patent Application Publication
(10) Pub. No.: US 2006/0178662 A1
(43) Pub. Date: Aug. 10, 2006

(54) Warming gradient control for a cryoablation applicator

(76) Inventors: Kenneth L. Ripley, Virginia Beach, VA (US); Gregory M. Ayers, San Diego, CA (US); David J. Lentz, La Jolla, CA (US)

Correspondence Address: NEIL K. NYDEGGER;NYDEGGER & ASSOCIATES, 348 Olive Street, San Diego, CA, 92103, US

(21) Appl. No.: 11/050,974

(22) Filed: Feb. 4, 2005

(51) Int. Cl. A61B 18/02

(52) U.S. Cl. 606/21; 606/23

(57) ABSTRACT

A method for effectively cryoablating tissue cells includes a regimen of selected cooling and warming rates. Specifically, cells are typically ablated by first cooling the cells at a relatively fast cooling rate (e.g. greater than 200° C. per minute) to reduce the cell temperature to below a minimum temperature (e.g. minus 10-15° C.) required to cause the cells to freeze. Next, the cells are thawed using a controlled, relatively slow warming rate (e.g. less than 100° C. per minute). The relatively fast cooling rate can cause intracellular and extra-cellular freezing of the tissue cells and the formation of relatively small ice crystals. Subsequently, during warming at a relatively slow warming rate, the small ice crystals can recrystallize and grow, causing a relatively high rate of cell destruction.

### FIELD OF THE INVENTION

The present invention pertains generally to systems and methods for effectively cryoablating target tissue. More particularly, the present invention pertains to methods for cryoablating tissue using specific freezing/thawing regimens. The present invention is particularly, but not exclusively, useful as a method for cryoablating tissue using a relatively fast cooling rate followed by a controlled relatively slow warming rate to minimize the probability of tissue cell survival.

### BACKGROUND OF THE INVENTION

It is well known that some (if not all) types of tissue cells can remain viable after being frozen and subsequently thawed. Indeed, tissue cells are often frozen to preserve the cells and research is ongoing with the ultimate goal that someday, whole organs may be completely and effectively preserved by freezing. Such an achievement would, hopefully, increase the transplant success rate and prolong the storage life of major organs prior to transplantation.

On the other hand, when the aim is to destroy diseased tissue, for example by using cryoablation techniques, cell survivability poses a serious obstacle. In fact, for almost all cryoablation procedures, the objective is complete ablation (i.e. destruction) of the targeted tissue. For example, it is often desirable to cryoablate internal tissue in a relatively non-invasive procedure. For this purpose, cryocatheters have been developed, such as the cryocatheter and associated refrigeration system that is disclosed in co-pending U.S. patent application Ser. No. 10/243,997, entitled “A Refrigeration Source for a Cryoablation Catheter.” Co-pending U.S. application Ser. No. 10/243,997 was filed on Sep. 12, 2002, is assigned to the same assignee as the present invention, and is hereby incorporated by reference herein. In one exemplary application of a cryocatheter, conduction blocks can be created that are particularly effective for curing heart arrhythmias, such as atrial fibrillation.

In a typical cryocatheter procedure, a cryoelement located at the distal end of the applicator is positioned near or in contact with the tissue requiring ablation (i.e. the target tissue). Next, a fluid refrigerant is expanded within the cryoelement, cooling the cryoelement to a cryogenic temperature to thereby cryoablate the target tissue. Heretofore, the standard practice has been to continue the expansion of refrigerant in the cryoelement to maintain the tissue in a frozen state for a predetermined residence time (e.g. 5 minutes). At the completion of the residence time, the standard practice has been to discontinue the expansion of refrigerant inside the cryoelement, allowing the tissue to passively warm and thaw at a rate dictated by the absorption rate of surrounding body heat by the affected tissue. Since little attention has been directed toward controlling the cooling or warming rates, the goal of complete target tissue destruction has not always been obtained.

In addition to cryocatheters, exposed tissue can be also destroyed using a cryoprobe. For example, a suitable cryoprobe and associated refrigeration system for destroying exposed tissue is disclosed in co-pending U.S. patent application Ser. No. 10/646,486, entitled “Reshapeable Tip for a Cryoprobe.” Co-pending U.S. application Ser. No. 10/646,486 was filed on Aug. 22, 2003, is assigned to the same assignee as the present invention, and is hereby incorporated by reference herein.

Experiment has shown that there are effectively at least three mechanisms, operable during cooling, which are responsible for causing cell death when tissue is frozen. The two main mechanisms are referred to, hereinafter, respectively, as “solution effects” and “intracellular freezing”. For most cell types, the percentage of cells which survive the cooling step is dependent on the cooling rate used to freeze the tissue and the lowest tissue temperature obtained during cooling. Moreover, some tissue cells exhibit a maximum cell survivability percentage at a certain cooling rate. As detailed further below, a portion of cells that survive the cooling step may be subsequently killed when the surviving cell is warmed to its original temperature.

The so-called “solution effects” result from four identifiable phenomena that occur simultaneously during freezing. These phenomena are: 1) a dehydration of the cell; 2) the concentration of solutes; 3) a decrease in cell size; and 4) the precipitation of solutes. On the other hand, as the name implies, “intracellular freezing” results in the freezing of water inside a tissue cell. Although water will freeze inside a tissue cell in both instances, it has been observed that if the “solution effects” predominate, there will be less water inside the cell to be frozen. This is due to dehydration and diminished cell size during freezing.

As a general proposition, it can be said that “solution effects” will predominate when the freezing velocity (i.e. the cooling rate) of tissue cells is relatively slow, and the cell permeability to water is high. On the other hand, “intracellular freezing” will predominate when the cooling rate is relatively fast, and the cell permeability to water is low. Moreover, it has also been observed that when the cooling rate is relatively fast and “intracellular freezing” predominates, relatively small ice crystals will form in the frozen water. This factor becomes important when the warming of the frozen tissue cells is considered.

During the warming of frozen tissue cells, it can happen that smaller ice crystals tend to experience a grain growth phenomenon referred to as “recrystallization”. This phenomenon occurs due to the high surface free energies of the small ice crystals, and results in the creation of larger crystals. Importantly for the present invention, it has been observed that recrystallization is most pronounced when tissue cells are warmed relatively slowly. It has been further observed that there is a higher probability the tissue cells will not survive when significant recrystallization is allowed to occur than when recrystallization is minimal or absent.

In light of the above, it is an object of the present invention to provide systems and methods suitable for the purposes of effectively cryoablating target tissue. It is another object of the present invention to provide systems and methods for cryoablating target tissue using specific freezing/thawing regimens which minimize the probability of tissue cell survival. It is yet another object of the present invention to provide systems and methods for controlling a cryocatheter to effectuate a pre-selected regimen of tissue cooling and warming rates. Yet another object of the present invention is to provide systems and methods for cryoablating target tissue which are easy to use, relatively simple to implement, and comparatively cost effective.

### SUMMARY OF THE INVENTION

The present invention is directed to systems and methods for cryoablating tissue cells. More specifically, the invention is directed to the cryoablation of tissue cells that can be characterized by a relationship of cooling rate versus cell survivability percentage that exhibits a maximum cell survivability percentage at a cooling rate, RMAX.

While it is not necessarily intended that all aspects of the present invention be limited by any one theory or mechanism of cryoablation, for tissue cells exhibiting a maximum cell survivability percentage at a cooling rate, RMAx, the invention recognizes that effective cell cryoablation can be achieved using a regimen of selected cooling and warming rates. Specifically, for the invention, cells are typically ablated by first cooling the cells at a relatively fast cooling rate (e.g. greater than 200° C. per minute delivered to the tissue) and subsequently allowing the cells to warm at a controlled, relatively slow warming rate (e.g. less than 100° C. per minute). In general, this warming rate is slower than the rate of passive warming as might occur when cooling is abruptly removed.

During cooling, the temperature of the cells is reduced to below the minimum temperature (e.g. minus 10-15° C.) required to cause the cells to freeze. More typically, the cells are cooled to a temperature (e.g. minus 70° C. to minus 80° C. at the tissue surface) that is substantially below the minimum freezing temperature. In one aspect of the invention, the cells are cooled at a rate greater than the rate, R_{MAX}, (where the maximum cell survivability percentage occurs) causing intracellular freezing of the tissue cells and the formation of relatively small ice crystals. Subsequently, during warming at a relatively slow warming rate, the small ice crystals recrystallize and grow, causing a relatively high rate of cell destruction. The result is an effective way to cryoablate the tissue cells with a relatively low probability of cell survival. Moreover, the cooling/warming cycle can be repeated, as desired, to further decrease the probability of cell survival.

Operationally, the methods of the invention are typically performed using an applicator, such as a probe or catheter, having a cryoelement positioned at the applicator's distal end. Typically, the cryoelement is formed with an expansion chamber to allow a fluid refrigerant to expand therein and cool the cryoelement. Supply and return lines are placed in fluid communication with the expansion chamber to respectively deliver a fluid refrigerant to the chamber for expansion therein and exhaust the expanded refrigerant therefrom.

In use, the cryoelement is first positioned proximate the target tissue (i.e. in contact with or close enough to the target tissue to cause a significant, measurable change in target tissue temperature in response to a change in cryoelement temperature). Once the cryoelement is proximate the target tissue, refrigerant is delivered to and expanded in the expansion chamber to cool the cryoelement and target tissue. Refrigerant then flows out of the chamber through the return line. In one implementation, the flow of coolant through the chamber is varied to achieve the cooling and warming rates described above. For example, a control valve operable on the supply line can be selectively adjusted to vary the flow of coolant through the chamber. Typically, during the cooling stage, a pre-selected, substantially constant flow of coolant is maintained. On the other hand, during the warming stage, the flow of coolant is slowly reduced until the tissue cells have warmed to a pre-selected temperature.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a perspective view of one exemplary implementation of a system for cryoablating internal target tissue shown operationally positioned in a patient;

FIG. 2 is a cross-sectional view of a distal portion of the cryoablation system shown in FIG. 1 as seen along line 2-2 in FIG. 1, with the restriction tube shown without sectioning for clarity;

FIG. 3 is a side view of a distal portion of the cryoablation system shown positioned at a treatment site in the vasculature of a patient; and

FIG. 4 is a graphical illustration showing the relationship between the cooling rate and the percentage of cells which survive the cooling step for an exemplary type of tissue cell.

### DESCRIPTION OF THE PREFERRED EMBODIMENTS

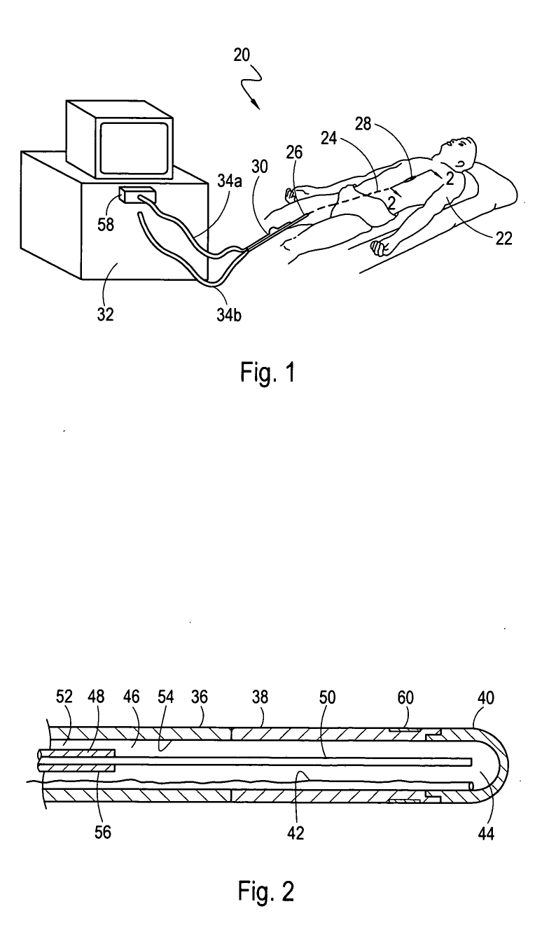

Referring initially to FIG. 1, a system 20 for ablating internal target tissue of a patient 22 is shown. As shown, the system 20 includes an applicator, which for the embodiment shown is a catheter 24. Although the system 20 is described herein for a catheter 24, those skilled in the pertinent art will appreciate that these methods can be implemented with other applicators such as a cryoprobe (not shown) that is configured to contact and ablate exposed tissue.

FIG. 1 shows that the catheter 24 extends from a proximal end 26, that remains outside the patient's body during a procedure, to a distal end 28. From FIG. 1 it can also be seen that the distal end 28 of the catheter 24 has been inserted into the patient 22 through a peripheral vein, such as the femoral vein, and advanced through the patient's vasculature until the distal end 28 has been positioned in the upper body of the patient 22. FIG. 1 further shows that the proximal end 26 of the catheter 24 is connected to a catheter handle 30, which in turn is connected to a fluid refrigerant supply unit 32 via a supply line umbilical 34a and a return line umbilical 34b. Although the system 20 is capable of performing a cryoablation procedure in an upper body vessel, such as a pulmonary vein, those skilled in the pertinent art will quickly recognize that the use of the system 20, as herein described, is not limited to use in any one type of vessel, but, instead can be used throughout the human body in vascular conduits and other ductal systems, or by direct application to a target tissue.

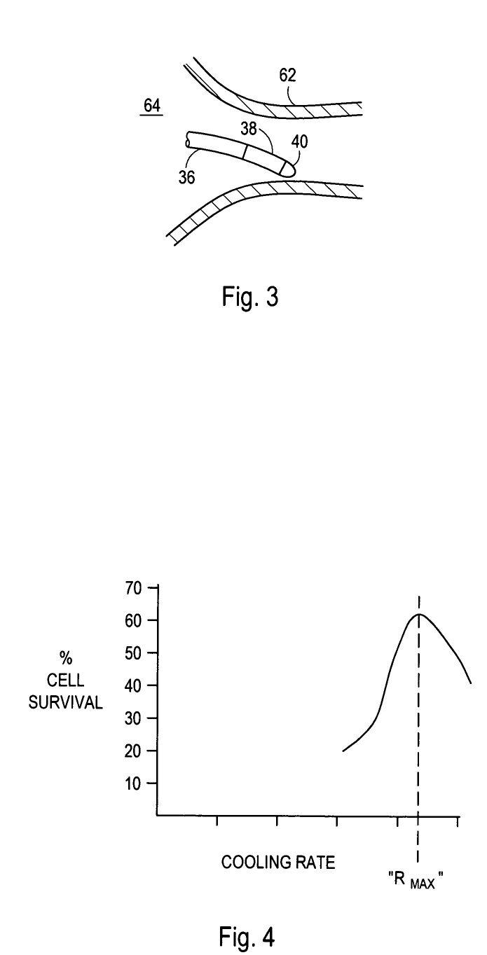

Turning now to FIG. 2, the cryotip (i.e. the distal portion) of the catheter 24 is shown in greater detail. Referring to FIG. 2 and proceeding along the catheter 24 in a proximal to distal direction, it can be seen that the catheter 24 includes a hollow, cylindrical catheter tube 36, an optional articulation segment 38, and a cryoelement 40. The articulation segment 38 can be used to both steer the catheter 24 during an advancement of the distal end 28 of the catheter 24 through body conduits, and to place the cryoelement 40 proximate to the target tissue (see FIG. 3). A suitable articulation segment for use in the system 20 is disclosed in co-pending, co-owned U.S. patent application Ser. No. 10/223,077, filed on Aug. 16, 2002, and titled “Catheter Having Articulation System”. It can also be seen from FIG. 2 that the system 20 includes a pull wire 42 which is attached to the cryoelement 40 and extends to an extracorporeal location (e.g. the handle 30) where the pull wire 42 can be manipulated to selectively reshape the articulation segment 38.

Continuing now with reference to FIG. 2, it can be seen that the cryoelement 40 is formed with an expansion chamber 44 that is placed in fluid communication with the lumen 46 of the catheter tube 36. Cross-referencing FIGS. 1 and 2, it will be appreciated that the system 20 includes a supply line which includes the supply line umbilical 34a, a supply tube 48 and a restriction tube 50 (e.g. capillary tube) that is positioned at the distal end of the supply tube 48. As shown, the supply tube 48 is positioned in the lumen 46 of the catheter tube 36 and placed in fluid communication with the umbilical 34a. It can be further seen that the supply tube 48 is positioned inside the lumen 46 of the catheter tube 36 to establish a return line 52 between the inner surface 54 of the catheter tube 36 and the outer surface 56 of the supply tube 48. For the system 20, the return line 52 is placed in fluid communication with the return line umbilical 34b.

Continuing with cross reference to FIGS. 1 and 2, it can be seen that system 20 further includes an adjustable control valve 58 configured to control the pressure in (and flow of refrigerant through) the supply line. With this cooperation of structure, fluid refrigerant from the refrigerant supply unit 32 passes through the valve 58 and into the supply line umbilical 34a. From the umbilical 34a, the fluid refrigerant passes through the handle 30 and into the supply tube 48. The fluid refrigerant then traverses the supply tube 48 and flows into the restriction tube 50. Fluid refrigerant then exits the distal end of the restriction tube 50 and expands into the chamber 44 to cool the cryoelement 40.

In one embodiment of the present invention, a fluid refrigerant is used that transitions from a liquid state to a gaseous state as it expands into the expansion chamber 44 of the cryoelement 40. A suitable refrigerant supply unit 32 for delivering a refrigerant in a liquid state to the distal end of the restriction tube 50 for transition to a gaseous state in the expansion chamber 44 is disclosed in co-pending, co-owned U.S. patent application Ser. No. 10/243,997, entitled “A Refrigeration Source for a Cryoablation Catheter” and filed on Sep. 12, 2002. Co-pending U.S. patent application Ser. No. 10/243,997 was previously incorporated by reference herein. Heat absorbed by the refrigerant during the liquid to gas phase transition (i.e. latent heat) cools the cryoelement 40. After expansion, the gaseous fluid refrigerant passes through the return line 52 and exits at the proximal end 26 of the cryocatheter 24. In one implementation, nitrous oxide is used as the refrigerant with suction applied to the return line 52 allowing the cryoelement 40 to be cooled to a temperature of approximately −85 degrees Celsius. For the system 20, the cryoelement 40 is made of a thermally conductive material (e.g. metal) to allow heat to flow easily between the chamber 44 and the target tissue. FIG. 2 further shows that the catheter 24 can include one or more electrode bands 60, which can be used alone or in conjunction with the conductive cryoelement 40 to map electrical signals of the heart. Those skilled in the pertinent art will appreciate that the cryotip can include other structures (not shown), including sensors, such as one or more pressure sensors or thermocouples, for use in measuring and controlling the temperature of the cryotip.

### Operation

As best seen by cross-referencing FIG. 1 with FIG. 3, in a typical cryoablation procedure using the system 20, the cryoelement 40 is initially inserted into a body conduit of a patient 22 (e.g. vasculature) and then advanced through the conduit using the catheter tube 36 and handle 30 until the cryoelement 40 is located proximate the target tissue. For example, FIG. 3 illustrates an exemplary application in which the cryoelement 40 has been positioned proximate to tissue surrounding an ostium where a pulmonary vein 62 connects with the left atrium 64. The skilled artisan will appreciate that this tissue can be cryoablated to form a conduction block as a treatment for heart arrhythmias, such as atrial fibrillation.

For the present methods, effective cell cryoablation is achieved using a regimen of selected cooling and warming rates. Specifically, as shown in FIG. 4, certain tissue cells can be characterized by a relationship of cooling rate versus cell survivability percentage that exhibits a maximum cell survivability percentage at a cooling rate, R_{MAX}. For example, the cells can be cooled at a rate greater than the rate, R_{MAX}, (where the maximum cell survivability percentage occurs) causing intracellular freezing of the tissue cells and the formation of relatively small ice crystals. The size and type of cells that are to be cryoablated are variables that may be considered when determining an effective cooling rate. Additionally, the ice ball that is created during cooling at the target site will affect the cell cooling and cell warming rates. During cooling, the temperature of the cells is reduced to below the minimum temperature (e.g. minus 10-15° C.) required to cause the cells to freeze. More typically, the cells are cooled to a temperature (e.g. minus 70° C. to minus 80° C.) that is substantially below the minimum freezing temperature of the cells.

After intracellular freezing, the cells are warmed at a relatively slow warming rate, causing the small ice crystals to recrystallize and grow. This process leads to a relatively high rate of cell destruction. The result is an effective way to cryoablate the tissue cells with a relatively low probability of cell survival. In a typical implementation, cells are ablated by first cooling the cells at a relatively fast cooling rate (e.g. greater than 200° C. per minute at the tissue surface) and subsequently allowing the cooled cells to warm at a controlled, relatively slow warming rate (e.g. less than 100° C. per minute). In some applications, a warming rate of less than 50° C. per minute is used, while other applications are performed using a warming rate between 10-50° C. per minute. In certain cases, some tissue cells are destroyed by thrombosis of the included microcirculation (i.e. starvation or suffocation).

One way to effectuate the cooling/warming regimen described above is to vary the flow of fluid refrigerant in the supply line using the valve 58. Once the cryoelement 40 is proximate the target tissue as shown in FIG. 3, refrigerant is delivered to and expanded in the expansion chamber 44 (see FIG. 2) to cool the cryoelement 40 and target tissue. Refrigerant then flows out of the chamber 44 through the return line 52. By adjusting the valve 58, the flow of coolant through the chamber 44 can be varied to achieve the controlled cooling and warming rates described above. Typically, during the cooling stage, a pre-selected, substantially constant flow of coolant through the chamber 44 is maintained. On the other hand, during the warming stage, the flow of fluid refrigerant through the chamber 44 is slowly reduced by selectively adjusting the valve 58 to achieve a pre-selected, controlled warming rate. Warming at the controlled rate is continued until the tissue cells have warmed to a pre-selected temperature. Moreover, the cooling/warming cycle can be repeated, as desired, to further decrease the probability of cell survival. Once adequate cryoablation has been achieved, the cryoelement 40 is removed from the patient 22 to complete the procedure.

While the particular Warming Gradient Control For A Cryoablation Applicator and corresponding methods of use as herein shown and disclosed in detail are fully capable of obtaining the objects and providing the advantages herein before stated, it is to be understood that they are merely illustrative of the presently preferred embodiments of the invention and that no limitations are intended to the details of construction or design herein shown other than as described in the appended claims.

What is claimed is:

1. A method for cryoablating in-situ tissue cells, the method comprising the steps of:
placing a distal tip of an applicator in contact with a target tissue of a patient;
flowing a fluid refrigerant through the distal tip to cool the target tissue cells at a cooling rate sufficient to cause intracellular and extra-cellular freezing of the cells and generate ice crystals; and thereafter
reducing the flow of fluid refrigerant through the distal tip to warm the tissue cells at a controlled warming rate to recrystallize the ice crystals and cryoablate the tissue cells.

2. A method as recited in claim 1 wherein the flowing step cools the tissue cells to below minus 10° C.

3. A method as recited in claim 1 wherein the flowing step cools the tissue cells at a cooling rate greater than 200° C. per minute.

4. A method as recited in claim 1 wherein the reducing step warms the tissue cells at a warming rate less than 100° C. per minute.

5. A method as recited in claim 1 wherein the method further comprises the step of repeating said flowing and reducing steps to cryoablate additional tissue cells.

6. A method as recited in claim 1 wherein tissue cells of a pulmonary vein are cryoablated to treat atrial fibrillation.

7. A method for cryoablating in-situ tissue cells, the tissue being characterized by a relationship of cooling rate versus cell survivability percentage that exhibits a maximum cell survivability percentage at a cooling rate, R_{MAX}, the method comprising the steps of:
providing an applicator having a cryoelement;
placing the cryoelement proximate the tissue cells;
flowing a fluid refrigerant through the cryoelement to cool the tissue cells at a cooling rate greater than the cooling rate, R_{MAX}, to freeze the tissue cells; and thereafter
reducing the flow of fluid refrigerant through the cryoelement to warm the tissue cells at a controlled warming rate to cryoablate tissue cells.

8. A method as recited in claim 7 wherein the flowing step cools the tissue cells to below minus 10° C.

9. A method as recited in claim 7 wherein the flowing step cools the tissue cells at a cooling rate greater than 200° C. per minute.

10. A method as recited in claim 7 wherein the reducing step warms the tissue cells at a warming rate less than 100° C. per minute.

11. A method as recited in claim 7 wherein the method further comprises the step of repeating said flowing and reducing steps to cryoablate additional tissue cells.

12. A method as recited in claim 7 wherein the flowing step freezes issue cells by intracellular freezing.

13. A method as recited in claim 12 wherein the reducing step cryoablates tissue cells by recrystallization.

14. A method as recited in claim 12 wherein the tissue cells have an included microcirculation and the reducing step destroys the tissue cells by thrombosis of the included microcirculation.

15. A system for cryoablating target tissue cells, the system comprising:
a cryoelement;
a means for delivering the cryoelement to a location proximate the target tissue cells;
a means for flowing a fluid refrigerant through the cryoelement to cool the tissue cells at a cooling rate sufficient to cause intracellular and extra-cellular freezing of the cells and generate ice crystals; and
a means for reducing the flow of fluid refrigerant through the cryoelement to warm the frozen tissue cells at a controlled warming rate to recrystallize the ice crystals and cryoablate tissue cells.

16. A system as recited in claim 15 wherein the flowing means cools the tissue cells to below minus 10° C.

17. A system as recited in claim 15 wherein the flowing means cools the tissue cells at a cooling rate greater than 200° C. per minute.

18. A system as recited in claim 15 wherein the reducing means warms the tissue cells at a warming rate less than 100° C. per minute.

19. A system as recited in claim 15 wherein the delivering means is a catheter having a supply line to deliver flowing refrigerant to the cryoelement.

20. A system as recited in claim 19 wherein the reducing means comprises an adjustable control valve operable on the supply line.
